(19) United States
(12) Patent Application Publication
(10) Pub. No.: US 2024/0277772 A1
(43) Pub. Date: Aug. 22, 2024

(54) COMBINED ADMINISTRATION COMPOSITION FOR PREVENTING OR TREATING ATOPIC DERMATITIS, COMPRISING FUNCTION-ENHANCED STEM CELLS AND REGULATORY T CELLS

(71) Applicants: SCM LIFESCIENCE CO., LTD., Incheon (KR); TERAIMMUNE, LLC, Germantown, MD (US)

(72) Inventors: Sun Uk SONG, Incheon (KR); Si Na KIM, Incheon (KR); Jeong Hyun MOON, Incheon (KR); Jeong Heon YOON, Gaithersburg, MD (US); Na Ri BYUN, Gaithersburg, MD (US)

(73) Assignees: SCM LIFESCIENCE CO., LTD., Incheon (KR); TERAIMMUNE, LLC, Germantown, MD (US)

(21) Appl. No.: 18/566,349

(22) PCT Filed: May 30, 2022
(86) PCT No.: PCT/KR2022/007696

§ 371 Date: Dec. 20, 2023

(30) Foreign Application Priority Data
Jun. 1, 2021 (KR) 10-2021-0070810

(51) Int. Cl. A61K 35/28; A61K 8/98; A61K 35/17; A61K 39/00; A61P 17/04; A61P 37/06; A61Q 19/00; C12N 5/0775

(52) U.S. Cl. CPC A61K 35/28; A61K 8/981; A61K 35/17; A61K 39/4611; A61K 39/4621; A61K 39/46433; A61P 17/04; A61P 37/06; A61Q 19/00; C12N 5/0663; C12N 2500/24; C12N 2500/38; C12N 2501/25

(57) ABSTRACT

The present invention relates to: a composition and a kit for preventing, alleviating or treating atopic dermatitis, comprising primed function-enhanced stem cells and regulatory T cells; and a method for preventing, alleviating or treating atopic dermatitis by using same. When function-enhanced stem cells, of the present invention, treated with TNF-α, IFN-γ and IFN-α or TNF-α, IFN-γ, IFN-α and vitamins are treated in combination with regulatory T cells, the imbalance of Th1/Th2 cells of an atopic dermatitis patient can be alleviated and the status of the atopic dermatitis patient can be shifted to Th1, and thus the present invention can be widely used in various fields of prevention, alleviation or treatment of atopic dermatitis.

### TECHNICAL FIELD

The present invention relates to a composition and a kit for preventing, alleviating, or treating atopic dermatitis including primed function-enhanced stem cells and regulatory T cells, and a method for preventing, alleviating or treating atopic dermatitis by using same.

### BACKGROUND ART

Stem cells are undifferentiated cells, which can divide for a long time through self-renewal and refer to cells capable of differentiating into various types of cells under a specific environment. The stem cells may be divided into embryonic stem cells and adult stem cells depending on an originating tissue, but therapeutic experiments using the embryonic stem cells have difficulties due to ethical aspects and tumorigenicity, whereas the adult stem cells have an advantage of being easily obtained from various tissues, and thus, studies are being actively conducted to apply the stem cells to the treatment of various diseases.

To date, many compound immunosuppressants or anti-inflammatory agents have been developed, and the most clinically commonly used immunosuppressants include cyclosporine (Neoral, Cipol A), azathioprine (imuran), prednisolone (a kind of steroid), and the like. The immunosuppressant inhibits several processes, such as phagocytosis of antigens by macrophages, antigen recognition by lymphocytes, cell division, division of T cells and B cells, and antibody production, during a process from antigen stimulation to antibody production, thereby causing immunosuppression. Most of these drugs have anti-tumor activity at the same time, but the reason is that these drugs inhibit cell division through DNA disorders, inhibition of DNA synthesis, and the like. However, representative side effects according thereto include hypertension and nephrotoxicity (decreased kidney function), and since the incidence of these side effects is high, there are problems such as a need to sufficiently observe the progress in use. Other side effects rarely include trembles, seizure, hepatitis, bile retention, increased uric acid in the blood, decreased muscle strength, hypertrichosis, and gingival hypertrophy. Among the commonly used inhibitors, azathioprine inhibits bone marrow functions such as decreased white blood cell count, anemia, and decreased platelets, and may have complications such as pancreatitis, hepatitis, and bile retention, and rarely hair loss and fever. Prednisolone, one of the steroid agents, started to be first used among immunosuppressants, but is a drug to be careful about not only promoting arteriosclerosis, but also causing hypertension, gastric ulcer, diabetes, growth inhibition, osteoporosis, cataract, and glaucoma. Therefore, the need for safe immunosuppressants or anti-inflammatory agents is emerging.

In order to solve these problems, therapies using stem cells have recently been attempted for the treatment of inflammatory and immune diseases. In particular, mesenchymal stem cells (MSCs) are known to suppress inflammation, induce the generation of regulatory T cells (Treg), or induce the death of immune cells involved in apoptosis, and thus, the development of various therapeutic agents using the MSCs is being actively conducted.

However, in various clinical trials using stem cells, there has been reported a problem that persistence of effects or in vivo survival rates as a result of long-term follow-up was extremely low, and thus, various attempts are being made to improve stem cell therapeutics. For example, in order to increase the therapeutic effect, there are attempts of methods of pre-processing various chemicals and peptides capable of inserting specific genes into stem cells or boosting stem cell functions, and methods of adding stimulation conditions such as hypoxia, temperature, and light during culture, or in order to increase the viability of stem cells, methods of administering the stem cells together with a support have been studied. Among various studies for enhancing the stem cell functions, a method of improving functions using genetic manipulation may be effective, but problems such as the safety of genes to be introduced and the like are pointed out.

Therefore, various studies have been attempted to enhance the intrinsic functions of stem cells through treatment of various priming factors during culture rather than enhancing the functions by introducing the genes into the stem cells themselves.

Meanwhile, regulatory T cells (Tregs) are a type of T cells that play an important role in naturally preventing excessive inflammation and immune responses in our body. However, in cases of autoimmune diseases and chronic inflammatory diseases, the functions and number of regulatory T cells are significantly reduced. Although the exact cause of atopic dermatitis is not yet clear, it is known to be triggered by an inappropriate T helper 2 (Th2) cell response to environmental allergens, which depends on an individual's genetic factors. The Th2 cells increase the concentration of immunoglobulin E (IgE) in the serum, and the increased IgE activates mast cells to cause atopic dermatitis. Additionally, it is believed that defective responses of Th1 cells can also contribute to the worsening of atopic dermatitis. Accordingly, research has been conducted to treat atopic dermatitis by controlling the imbalance between Th1 and Th2 cells through functional antagonism as a strategy for treating atopic dermatitis. Recently, researchers have been trying to regulate the imbalance of Th1/Th2 cells by promoting a Th1 response and suppressing a Th2 response through regulation of T helper cell differentiation with pathogen-related molecules, such as bacterial CpG motif or toll-like receptor 9 ligands.

There haven't been many studies conducted on a treatment method for atopic dermatitis that focuses on controlling the immune imbalance. Specifically, in using stem cells to treat Th2-derived atopic dermatitis, there are limited reports of treatments that can shift to Th1 responses in the patients.

### DISCLOSURE

### Technical Problem

The present inventors confirmed that an effect of preventing or treating atopic dermatitis of stem cells may be significantly enhanced by treating TNF-α, IFN-γ and IFN-α; or TNF-α, IFN-γ, IFN-α and vitamins while conducting research to further enhance the stem cell functions, confirmed that when treated in combination with regulatory T cells, a better atopic dermatitis therapeutic effect was exhibited, and then completed the present invention.

Therefore, an object of the present invention is to provide a composition and a kit for preventing, alleviating or treating atopic dermatitis, including stem cells primed with TNF-α, IFN-γ and IFN-α; or TNF-α, IFN-γ, IFN-α and vitamins and regulatory T cells; and a method for treating atopic dermatitis by using same.

### Technical Solution

In order to achieve the object, one aspect of the present invention provides a pharmaceutical composition for preventing or treating atopic dermatitis, including stem cells treated with TNF-α, IFN-γ, and IFN-α and regulatory T cells.

Further, the present invention provides a pharmaceutical composition for preventing or treating atopic dermatitis, including stem cells treated with TNF-α, IFN-γ, IFN-α, and at least one vitamin selected from the group consisting of vitamin B2, vitamin B3, vitamin B5 and vitamin B6 and regulatory T cells.

Further, another aspect of the present invention provides a kit for preventing or treating atopic dermatitis including the composition.

Further, yet another aspect of the present invention provides a combined administration kit for preventing or treating atopic dermatitis, including compartment 1 containing TNF-α, IFN-γ and IFN-α for priming stem cells; compartment 2 containing stem cells; and compartment 3 containing regulatory T cells.

Further, the present invention provides a combined administration kit for preventing or treating atopic dermatitis, including compartment 1 containing TNF-α, IFN-γ, IFN-α, and at least one vitamin selected from the group consisting of vitamin B2, vitamin B3, vitamin B5 and vitamin B6; compartment 2 containing stem cells; and compartment 3 containing regulatory T cells.

Further, the present invention provides a combined administration kit for preventing or treating atopic dermatitis, including compartment 1 containing stem cells treated with TNF-α, IFN-γ and IFN-α; and compartment 2 containing regulatory T cells.

Further, the present invention provides a combined administration kit for preventing or treating atopic dermatitis, including compartment 1 containing stem cells treated with TNF-α, IFN-γ, IFN-α, and at least one vitamin selected from the group consisting of vitamin B2, vitamin B3, vitamin B5 and vitamin B6; and compartment 2 containing regulatory T cells.

Further, yet another aspect of the present invention provides a cell therapy for preventing or treating atopic dermatitis, including a first composition containing stem cells treated with TNF-α, IFN-γ and IFN-α; and a second composition containing regulatory T cells.

Further, the present invention provides a cell therapy for preventing or treating atopic dermatitis, including a first composition containing stem cells treated with TNF-α, IFN-γ, IFN-α, and at least one vitamin selected from the group consisting of vitamin B2, vitamin B3, vitamin B5 and vitamin B6; and a second composition containing regulatory T cells.

Further, yet another aspect of the present invention provides a method for preventing or treating atopic dermatitis, including 1) administering stem cells treated with TNF-α, IFN-γ and IFN-α to a subject; and 2) administering regulatory T cells to the subject.

Further, the present invention provides a method for preventing or treating atopic dermatitis, including 1) administering stem cells treated with TNF-α, IFN-γ, IFN-α, and at least one vitamin selected from the group consisting of vitamin B2, vitamin B3, vitamin B5 and vitamin B6 to a subject; and 2) administering regulatory T cells to the subject.

Further, yet another aspect of the present invention provides a cosmetic composition for preventing or alleviating atopic dermatitis, including stem cells treated with TNF-α, IFN-γ, and IFN-α; and regulatory T cells.

Further, the present invention provides a cosmetic composition for preventing or alleviating atopic dermatitis, including stem cells treated with TNF-α, IFN-γ, IFN-α, and at least one vitamin selected from the group consisting of vitamin B2, vitamin B3, vitamin B5 and vitamin B6; and regulatory T cells.

### Advantageous Effects

According to the present invention, when function-enhanced stem cells treated with TNF-α, IFN-γ and IFN-α or TNF-α, IFN-γ, IFN-α and vitamins are treated in combination with regulatory T cells, the imbalance of Th1/Th2 cells of an atopic dermatitis patient can be alleviated and the status of the atopic dermatitis patient can be shifted to Th1, and thus the present invention can be widely used in various fields of prevention, alleviation or treatment of atopic dermatitis.

### DESCRIPTION OF DRAWINGS

FIG. 1 is a diagram illustrating results of confirming increased expression of IDO and TSG6 according to a treatment concentration of TNF-α (***P<0.001, ****P<0.0001, compared to the control (0 ng/ml)).

FIG. 2 is a diagram illustrating results of confirming increased expression of IDO and TSG6 according to a treatment concentration of IFN-γ (*P<0.05, ****P<0.0001, compared to the control (0 ng/ml)).

FIG. 3 is a diagram illustrating results of confirming increased expression of IDO and TSG6 according to a treatment concentration of IFN-α (*P<0.05, **P<0.01, ***P<0.001, ****P<0.0001, compared to the control (0 ng/ml)).

FIG. 4 is a diagram illustrating results of confirming increased expression of IDO and TSG6 according to compositions shown in Table 1 (****P<0.0001) (0: untreated control; 1: TNF-α (10 ng/ml); 2: IFN-γ (10 ng/ml); 3: IFN-α (20 ng/ml); 4: TNF-α (10 ng/ml)+IFN-γ (10 ng/ml); 5: TNF-α (10 ng/ml)+IFN-α (20 ng/ml); 6: IFN-γ (10 ng/ml)+IFN-α (20 ng/ml); 7: TNF-α (10 ng/ml)+IFN-γ (10 ng/ml)+IFN-α (20 ng/ml)).

FIG. 5 is a diagram illustrating results of confirming increased expression of IDO and TSG6 according to single treatment of vitamins shown in Table 2 (0: untreated control; 1: Vitamin A (10 μg/ml); 2: Vitamin B1 (50 μg/ml); 3: Vitamin B2 (5 μg/ml); 4: Vitamin B3 (50 μg/ml); 5: Vitamin B5 (50 μg/ml); 6: Vitamin B6 (50 μg/ml); 7: Vitamin B12 (50 μg/ml); 8: Vitamin D2 (10 μg/ml); 9: Vitamin D3 (10 μg/ml)).

FIG. 6 is a diagram illustrating results of confirming increased expression of IDO and TSG6 according to the treatment with compositions shown in Table 3 in which vitamins are added to TNF-α, IFN-γ, and IFN-α, respectively. (*P<0.05, **P<0.01, ***P<0.001, ****P<0.0001, compared to group 1) (0: untreated control; 1: TNF-α+IFN-γ+IFN-α; 2: addition of vitamin A to Experimental Group 1; 3: addition of vitamin B1 to Experimental Group 1; 4: addition of vitamin B2 to Experimental Group 1; 5: addition of vitamin B3 to Experimental Group 1; 6: addition of vitamin B5 to Experimental Group 1; 7: addition of vitamin B6 to Experimental Group 1; 8: addition of vitamin B12 to Experimental Group 1; 9: addition of vitamin D2 to Experimental Group 1; 10: addition of vitamin D3 to Experimental Group 1)

FIG. 7 is a diagram illustrating results of confirming increased expression of ICAM1 and VCAM according to the treatment with compositions shown in Table 3 in which vitamins are added to TNF-α, IFN-γ, and IFN-α, respectively. (*P<0.05, **P<0.01, ***P<0.001, ****P<0.0001, compared to group 1) (0: untreated control; 1: TNF-α+IFN-γ+IFN-α; 2: addition of vitamin A to Experimental Group 1; 3: addition of vitamin B1 to Experimental Group 1; 4: addition of vitamin B2 to Experimental Group 1; 5: addition of vitamin B3 to Experimental Group 1; 6: addition of vitamin B5 to Experimental Group 1; 7: addition of vitamin B6 to Experimental Group 1; 8: addition of vitamin B12 to Experimental Group 1; 9: addition of vitamin D2 to Experimental Group 1; 10: addition of vitamin D3 to Experimental Group 1)

FIG. 8 is a diagram illustrating results of confirming inflammatory cytokine IFN-γ and anti-inflammatory cytokine IL-10 by co-culturing PHA-stimulated PBMCs with pcMSC2 or untreated cMSC, in order to confirm immunomodulatory ability of stem cells pcMSC2 treated with TNF-α, IFN-γ, IFN-α and vitamin B6 (***P<0.001, ****P<0.0001, compared to cMSC, P1: negative control, not stimulated with PHA).

FIG. 9 is a diagram illustrating a gating strategy to sort-purify regulatory T cells (Tregs) and non-regulatory T cells (conventional T cells, Tconv) using a flow cytometry cell sorter and their purities after sorting.

FIG. 10 is a diagram illustrating the expression of transcription factors Foxp3 and Helios as the characteristics of ex vivo expanded regulatory T cells on day 12.

FIG. 11 is a diagram illustrating intracellular levels of IL-2, IL-4, IFN-γ, and IL-17A secreted from regulatory T cells (Tregs) upon stimulation in 12 days of culture.

FIG. 12 is a schematic diagram illustrating the administration cycle and timing of pcMSC2 and regulatory T cells (Tregs) in an atopic dermatitis animal model.

FIG. 13 is a diagram illustrating results of confirming changes in skin lesions, after administering an experimental substance corresponding to each group of G1: Naive, G2: Veh. (for pcMSC2), G3: Veh. (for Tregs), G4: pcMSC2, G5: Tregs, and G6: pcMSC2+Tregs (D44).

FIG. 14 is a diagram illustrating results of confirming changes in total IgG1 production in serum through ELISA assay according to administration of PBS (control for pcMSC2), cell stabilizer (control for Tregs), pcMSC2, Tregs, pcMSC2+Tregs (D44) in an atopic dermatitis animal model. (***P<0.001, ****P<0.0001 compared to Veh.)

FIG. 15 is a diagram illustrating results of statistical analysis (T-test) of changes in total IgG1 production according to administration of PBS (control for pcMSC2), cell stabilizer (control for Tregs), pcMSC2, Tregs, pcMSC2+Tregs (D44) in an atopic dermatitis animal model.

FIG. 16 is a diagram illustrating results of confirming changes in total IgG2a production in serum through ELISA assay according to administration of PBS (control for pcMSC2), cell stabilizer (control for Tregs), pcMSC2, Tregs, pcMSC2+Tregs (D44) in an atopic dermatitis animal model. (*P<0.05, **P<0.01, ****P<0.0001 compared to Veh.)

FIG. 17 is a diagram illustrating results of statistical analysis (T-test) of changes in total IgG2a production in serum according to administration of PBS (control for pcMSC2), cell stabilizer (control for Tregs), pcMSC2, Tregs, pcMSC2+Tregs (D44) in an atopic dermatitis animal model.

### BEST MODE OF THE INVENTION

Hereinafter, the present invention will be described in detail.

The present invention provides a pharmaceutical composition for preventing or treating atopic dermatitis, including stem cells treated with TNF-α, IFN-γ, IFN-α; or TNF-α, IFN-γ, IFN-α, and at least one vitamin selected from the group consisting of vitamin B2, vitamin B3, vitamin B5 and vitamin B6, and regulatory T cells.

As compared with stem cells untreated with TNF-α, IFN-γ and IFN-α; or TNF-α, IFN-γ, IFN-α, and at least one vitamin selected from the group consisting of vitamin B2, vitamin B3, vitamin B5 and vitamin B6, the stem cells of the present invention have excellent anti-inflammatory and immunomodulatory ability and thus may be effectively used for prevention or treatment of atopic dermatitis. Particularly, when treated in combination with regulatory T cells, a therapeutic effect on atopic dermatitis may be more significantly improved.

In the present invention, the stem cells treated with TNF-α, IFN-γ and IFN-α; or TNF-α, IFN-γ, IFN-α and at least one vitamin selected from the group consisting of vitamins B2, B3, B5, and B6 may be referred to as “primed stem cells” or “function-enhanced stem cells” and the “primed stem cells” and the “function-enhanced stem cells” may be used interchangeably. The primed stem cells refer to stem cells that exhibit excellent atopic dermatitis therapeutic effects by significantly increasing the immunomodulatory and inflammatory regulation ability of stem cells by treatment with the priming factors of the present invention.

In the present invention, the “priming factors” may mean a combination of TNF-α, IFN-γ, and IFN-α, or a combination of further including at least one vitamin selected from the group consisting of vitamin B2, vitamin B3, vitamin B5, and vitamin B6 in the combination.

The combination of the priming factors may exhibit a synergistic effect compared to treating stem cells with these single ingredients, and effectively induce enhanced functions of desired stem cells even at a low treatment concentration.

In the present invention, “function enhancement” refers to the enhancement of the inherent properties and effects of stem cells by treatment with the priming factors, and particularly, may preferably mean improvement of an effect of preventing, alleviating or treating atopic dermatitis.

Specifically, the primed stem cells of the present invention may be stem cells treated with TNF-α, IFN-γ, and IFN-α. More specifically, the TNF-α, IFN-γ, and IFN-α may be treated in a ratio of 0.1 to 3:0.1 to 3:0.1 to 3 (w/v), preferably 1:1:0.1 to 3 (w/v), more preferably 1:1:1 to 3 (w/v), and much more preferably 1:1:1 to 2.5 (w/v). In an embodiment of the present invention, the stem cells were treated and cultured with TNF-α, IFN-γ, and IFN-α at a combined concentration of 10 ng/ml, 10 ng/ml, and 20 ng/ml to confirm a therapeutic effect of the stem cells on atopic dermatitis.

In addition, the stem cells of the present invention may be stem cells further treated with vitamins in addition to the combination of TNF-α, IFN-γ, and IFN-α, and specifically, stem cells further treated with at least one vitamin selected from the group consisting of vitamin B2, vitamin B3, vitamin B5, and vitamin B6. When the stem cells are primed by treatment with a combination of priming factors further containing vitamins, the enhanced functions of the stem cells induced by treatment with TNF-α, IFN-γ and IFN-α may be more significantly improved, and the effect of preventing or treating atopic dermatitis may also be significantly improved. The TNF-α, IFN-γ, IFN-α and vitamins may be treated to the stem cells in a ratio of 0.1 to 3:0.1 to 3:0.1 to 3:100 to 10,000 (w/v), preferably 1:1:0.1 to 3:300 to 6,000 (w/v). More specifically, when vitamin B2 is added to TNF-α, IFN-γ, and IFN-α, TNF-α, IFN-γ, IFN-α and the vitamin may be treated in a ratio of 0.1 to 3:0.1 to 3:0.1 to 3:300 to 1,000 (w/v), more preferably 1:1:0.1 to 3:300 to 600 (w/v), much more preferably 1:1:0.1 to 3:400 to 550 (w/v), and even more preferably 1:1:2:500 (w/v). In addition, when vitamin B3, vitamin B5, or vitamin B6 is added to TNF-α, IFN-γ, and IFN-α, TNF-α, IFN-γ, IFN-α and the vitamin may be treated in a ratio of 0.1 to 3:0.1 to 3:0.1 to 3:1,000 to 10,000 (w/v), more preferably 0.1 to 3:0.1 to 3:0.1 to 3:1,000 to 6,000 (w/v), much more preferably 1:1:0.1 to 3:4,000 to 6,000 (w/v), even more preferably 1:1:0.1 to 3:4,000 to 5,500 (w/v), and even more preferably 1:1:2:5,000 (w/v).

In an embodiment of the present invention, as concentrations for combined treatment, 10 ng/ml of TNF-α, 10 ng/ml of IFN-γ, and 20 ng/ml of IFN-α were selected, and 5 μg/ml of vitamin B2, 50 μg/ml of vitamin B3, 50 μg/ml of vitamin B5 and 50 μg/ml of vitamin B6 were selected and treated to the stem cells to confirm the enhancement of immunomodulatory and inflammatory regulation functions. As a result, in Experimental Groups added with vitamins, compared to treatment of TNF-α, IFN-γ, and IFN-α, it was confirmed that not only the expression of IDO and TSG6 was increased, but also the expression of intercellular adhesion molecule 1 (ICAM1) and vascular cell adhesion molecule (VCAM) as adhesion factors expressed on the surface of stem cells was significantly increased.

In particular, in an embodiment of the present invention, it was confirmed that stem cells with an enhanced therapeutic effect on atopic dermatitis may be prepared by treatment with priming factors of a combination of TNF-α, IFN-γ, IFN-α, and vitamin B6, which was called “Primed clonal Mesenchymal Stem Cell 2 (pcMSC2).”

In the present invention, the ‘priming treatment’ or ‘treatment of priming factors’ may mean treating stem cells with a combination of TNF-α, IFN-γ, and IFN-α, or a combination of further containing at least one vitamin selected from the group consisting of vitamin B2, vitamin B3, vitamin B5, and vitamin B6 in the combination for 12 hours to 36 hours, preferably 20 hours to 25 hours, and then culturing the stem cells. For the culture, stem cell culture media widely known in the art may be used without limitation.

The primed stem cells function-enhanced by treatment of TNF-α, IFN-γ, and IFN-α; or TNF-α, IFN-γ, IFN-α, and the vitamins and the regulatory T cells included in the pharmaceutical composition of the present invention may be administrated sequentially or simultaneously in combination, respectively.

In the present invention, the primed stem cells function-enhanced by treatment with TNF-α, IFN-γ, and IFN-α; or TNF-α, IFN-γ, IFN-α and the vitamins refer to cells having ability to differentiate into two or more cells while having self-replication ability, and may be classified into totipotent stem cells, pluripotent stem cells, and multipotent stem cells. The stem cells may be appropriately selected without limitation depending on the purpose, and may be derived from adult cells, such as all known tissues, cells, and the like derived from mammals including humans, preferably humans, and for example, may be mesenchymal stem cells derived from fat, bone marrow, placenta (or placental tissue cells), or cord blood. In addition, the stem cells may refer to clonal stem cells.

The primed stem cells of the present invention may be stem cells enhanced with at least one selected from the group consisting of expression of TNFα-stimulated gene-6 (TSG6), expression of Indoleamine 2,3-dioxygenase (IDO), expression of Intercellular adhesion molecule 1 (ICAM1) and expression of Vascular cell adhesion molecule (VCAM) compared with stem cells untreated with the combination of the priming factors. The primed stem cells may exhibit at least one atopic dermatitis therapeutic effect selected from the group consisting of reduction of mast cells, reduction of total IgE, increased production of IgG2a, and reduction of histamine.

The present invention involves regulatory T cells that can be obtained from various sources such as blood, fat, bone marrow, placenta, or cord blood. It is preferable to isolate the regulatory T cells from the cord blood by selectively isolating only the CD4+ cells. The present invention describes regulatory T cells, which can be identified by being CD4⁺CD25^{+/high}CD127^{low} cells. The regulatory T cells of the present invention may be activated through CD3 and CD28 after isolation, and they can be stimulated with CD4⁻ cells derived from the same donor that were treated with mitomycin C on days 5 to 10 of the culture. The regulatory T cells of the present invention are not limited thereto, but can be cultured for 5 to 25 days or 10 to 20 days after isolation. These cultured regulatory T cells may maintain the characteristics of regulatory T cells without decreased immune functions that may occur in in-vitro culture.

The pharmaceutical composition of the present invention may further include suitable carriers, excipients, and diluents which are commonly used in the preparation of pharmaceutical composition. In addition, additives for solid or liquid formulations may be used in the preparation of the pharmaceutical composition. The additives for formulations may be either organic or inorganic.

Examples of the excipient may include lactose, sucrose, white sugar, glucose, corn starch, starch, talc, sorbitol, crystalline cellulose, dextrin, kaolin, calcium carbonate, silicon dioxide, and the like. Examples of the binder may include, for example, polyvinyl alcohol, polyvinyl ether, ethyl cellulose, methyl cellulose, arabic gum, tragacanth, gelatin, shellac, hydroxypropyl cellulose, hydroxypropyl methyl cellulose, calcium citrate, dextrin, pectin, and the like. Examples of the lubricant may include magnesium stearate, talc, polyethylene glycol, silica, hydrogenated vegetable oil, and the like. As a coloring agent, any coloring agent may be used as long as the coloring agent is permitted to be added to ordinary pharmaceuticals. These tablets and granules may be sugar-coated, gelatin-coated, or appropriately coated as needed. In addition, preservatives, antioxidants, etc. may be added as needed.

The pharmaceutical composition of the present invention may be prepared in any formulation commonly prepared in the art (e.g., Literature [Remington's Pharmaceutical Science, latest edition; Mack Publishing Company, Easton PA]), and the form of the formulation is not particularly limited, but may preferably be an external preparation. The external preparation of the present invention may include conventional external preparations, such as sheets, liquid coating agents, sprays, lotions, creams, cataplasmas, powders, penetration pads, sprays, gels, pastas, liniments, ointments, aerosols, powders, suspensions, and transdermal absorbents. These formulations are described in the literature [Remington's Pharmaceutical Science], a generally known prescription in all pharmaceutical chemistry.

The pharmaceutically effective dose of the present invention may vary depending on a patient's wound type, an application site, a treatment frequency, treatment time, a formulation, a patient's status, a type of adjuvant, and the like. The used amount is not particularly limited, but may be 0.00001 to 10000 μg when the daily effective dose of the pharmaceutical composition of the present invention is applied to a patient. The daily dose may be administered once a day, separately administered 2 to 3 times a day at appropriate intervals, or administered intermittently at intervals of several days. The dose of the stem cell therapeutic agent of the present invention may be preferably 1×10² to 1×10¹² cells/kg per day.

However, since the used amount of the pharmaceutical composition of the present invention is determined according to many related factors such as a route of administration, patient's age, sex, and weight, patient's severity, a type of wound, an application site, the number of treatments, a treatment time, a formulation, a patient's status, a type of adjuvant, and the like, the effective dose should not be understood to limit the scope of the present invention in any respect.

Further, the present invention provides a kit for preventing or treating atopic dermatitis including the pharmaceutical composition.

The kit of the present invention includes (a) a first composition containing stem cells treated with TNF-α, IFN-γ and IFN-α; or TNF-α, IFN-γ, IFN-α, and vitamins; and a second composition containing regulatory T cells, and the first composition and the second composition are each packaged in the form contained in separate containers or in one container divided into one or more compartments, and the vitamins may be one or more vitamins selected from the group consisting of vitamin B2, vitamin B3, vitamin B5, and vitamin B6.

Further, the present invention provides a combined administration kit for preventing or treating atopic dermatitis, including compartment 1 containing TNF-α, IFN-γ and IFN-α; or TNF-α, IFN-γ, IFN-α, and vitamins for priming the stem cells; compartment 2 containing stem cells; and compartment 3 containing regulatory T cells.

For treatment of atopic dermatitis, first, function-enhanced stem cells may be prepared by treating the stem cells in compartment 2 with TNF-α, IFN-γ and IFN-α; or TNF-α, IFN-γ, IFN-α, and vitamins for priming the stem cells in compartment 1, and administered simultaneously or sequentially in combination with the regulatory T cells in compartment 3 to prevent or treat atopic dermatitis.

Further, the present invention provides a combined administration kit for preventing or treating atopic dermatitis, including compartment 1 containing stem cells treated with TNF-α, IFN-γ and IFN-α; or TNF-α, IFN-γ, IFN-α, and vitamins; and compartment 2 containing regulatory T cells.

The stem cells included in compartment 1 may be stem cells function-enhanced by treatment with the priming factors of the present invention, such as TNF-α, IFN-γ, and IFN-α; or TNF-α, IFN-γ, IFN-α, and vitamins. For the treatment of atopic dermatitis, ingredients in compartment 1 or 2 may be administered simultaneously or sequentially to a subject in need of atopic dermatitis treatment.

In the kit, the vitamins may be one or more vitamins selected from the group consisting of vitamin B2, vitamin B3, vitamin B5, and vitamin B6.

Further, the present invention provides a cell therapy for preventing or treating atopic dermatitis, including a first composition containing stem cells treated with TNF-α, IFN-γ, and IFN-α; or TNF-α, IFN-γ, IFN-α, and vitamins; and a second composition containing regulatory T cells.

The cell therapy according to the present invention may be administered through various routes including oral, transdermal, subcutaneous, intravenous or intramuscular route, and the dose of the active ingredients may be appropriately selected according to several factors, such as a route of administration, the age, sex, and weight of a patient, and the severity of a patient. Preferably, the cell therapy may be administered parenterally, and may be administered by intravenous injection, subcutaneous injection, intradermal injection, intramuscular injection, intraperitoneal injection, and the like.

The first composition may preferably be for intravenous administration, and the second composition may be for intradermal administration. The composition for intravenous administration refers to a sterile composition in which a liquid drug is directly injected into the vein to act. The composition for intravenous administration includes all compositions that may be used in the manufacture of conventional injections, and include aqueous injections, non-aqueous injections, suspension injections, freeze-dried injections, etc., depending on a preparation method, but are not limited thereto. The second composition may be for intradermal administration, and the composition for intradermal administration refers to a composition for injecting a liquid drug in the form of an injection into the dermis, which is a layer immediately below the epidermis.

The injection order of the first composition and the second composition is not limited thereto, but the first composition may be injected and the second composition may also be injected, or the second composition may be injected and the first composition may be injected or simultaneously injected. The first composition and the second composition may be administered repeatedly as needed for the subject, and repeated administration intervals may be controlled depending on a patient's status, treatment conditions, or the like.

The kit of the present invention may be packaged in a form that includes separate containers containing each ingredient, or in a form that includes each ingredient in each compartment of a container divided into one or more compartments. The ingredients included in these separate containers or compartments may be administered simultaneously, separately, or sequentially to a patient in need thereof.

Further, the present invention provides a method for preventing or treating atopic dermatitis, including 1) administering stem cells treated with TNF-α, IFN-γ and IFN-α; or TNF-α, IFN-γ, IFN-α and vitamins to a subject in need thereof; and 2) administering regulatory T cells to the subject.

The administrations 1) and 2) may be sequentially or simultaneously performed.

The “sequentially” administration may be performed by step 2) after step 1), but step 1) after step 2).

The subject of the present invention may mean any animal, including humans. These animals may be mammals such as cattle, horse, sheep, pig, goat, camel, antelope, dog, and cat in need of treatment for similar symptoms to the atopic dermatitis as well as humans, or may be mammals excepting humans, but are not limited thereto. The subject may include all patients in need of treatment for atopic dermatitis, including patients undergoing treatment for atopic dermatitis, patients who have been treated for atopic dermatitis, and patients in need of treatment for atopic dermatitis.

In addition, the stem cells treated with TNF-α, IFN-γ and IFN-α; or TNF-α, IFN-γ, IFN-α and vitamins of the present invention may be treated in combination with existing drugs or treatment methods for treating atopic dermatitis. Particularly, when treated in combination with regulatory T cells like the present invention, a more significant therapeutic effect on atopic dermatitis may be achieved.

In the treatment method, the vitamins may be treated together with one or more vitamins selected from the group consisting of vitamin B2, vitamin B3, vitamin B5, and vitamin B6, and the TNF-α, IFN-γ and IFN-α may be treated in a ratio of 0.1 to 3:0.1 to 3:0.1 to 3 (w/v), preferably 1:1:0.1 to 3 (w/v), more preferably 1:1:1 to 3 (w/v), and much more preferably 1:1:1 to 2.5 (w/v).

In addition, the TNF-α, IFN-γ, IFN-α and vitamins may be treated to the stem cells in a ratio of 0.1 to 3:0.1 to 3:0.1 to 3:100 to 10,000 (w/v), preferably 1:1:0.1 to 3:300 to 6,000 (w/v). More specifically, when vitamin B2 is added to TNF-α, IFN-γ, and IFN-α, the TNF-α, IFN-γ, IFN-α and vitamin may be treated in a ratio of 0.1 to 3:0.1 to 3:0.1 to 3:300 to 1,000 (w/v), more preferably 1:1:0.1 to 3:300 to 600 (w/v), much more preferably 1:1:0.1 to 3:400 to 550 (w/v), and even more preferably 1:1:2:500 (w/v). In addition, when vitamin B3, vitamin B5, or vitamin B6 is added to TNF-α, IFN-γ, and IFN-α, the TNF-α, IFN-γ, IFN-α and the vitamin may be treated in a ratio of 0.1 to 3:0.1 to 3:0.1 to 3:1,000 to 10,000 (w/v), preferably 0.1 to 3:0.1 to 3:0.1 to 3:1,000 to 6,000 (w/v), more preferably 1:1:0.1 to 3:4,000 to 6,000 (w/v), much more preferably 1:1:0.1 to 3:4,000 to 5,500 (w/v), and even more preferably 1:1:2:5,000 (w/v).

Further, the present invention provides a cosmetic composition for preventing or alleviating atopic dermatitis, including stem cells treated with TNF-α, IFN-γ, and IFN-α and regulatory T cells.

More specifically, the TNF-α, IFN-γ, and IFN-α may be treated in a ratio of 0.1 to 3:0.1 to 3:0.1 to 3 (w/v), preferably 1:1:0.1 to 3 (w/v), more preferably 1:1:1 to 3 (w/v), and much more preferably 1:1:1 to 2.5 (w/v). In an embodiment of the present invention, the stem cells were treated and cultured with TNF-α, IFN-γ, and IFN-α at a combined concentration of 10 ng/ml, 10 ng/ml, and 20 ng/ml to be obtained, thereby confirming an effect of preventing or alleviating atopic dermatitis of the stem cells.

In addition, the stem cells of the present invention may be stem cells further treated with vitamins in addition to the combination of TNF-α, IFN-γ, and IFN-α, and specifically, stem cells further treated with at least one vitamin selected from the group consisting of vitamin B2, vitamin B3, vitamin B5, and vitamin B6.

When the stem cells are primed by treatment with a combination of priming factors further containing vitamins, the enhanced function of the stem cells induced by treatment with TNF-α, IFN-γ and IFN-α may be more significantly improved and the effect of preventing or alleviating atopic dermatitis may also be significantly improved. The TNF-α, IFN-γ, IFN-α and vitamins may be treated to the stem cells in a ratio of 0.1 to 3:0.1 to 3:0.1 to 3:100 to 10,000 (w/v), preferably 1:1:0.1 to 3:300 to 6,000 (w/v). More specifically, when vitamin B2 is added to TNF-α, IFN-γ, and IFN-α, the TNF-α, IFN-γ, IFN-α and the vitamin may be treated in a ratio of 0.1 to 3:0.1 to 3:0.1 to 3:300 to 1,000 (w/v), more preferably 1:1:0.1 to 3:300 to 600 (w/v), much more preferably 1:1:0.1 to 3:400 to 550 (w/v), and even more preferably 1:1:2:500 (w/v). In addition, when vitamin B3, vitamin B5, or vitamin B6 is added to TNF-α, IFN-γ, and IFN-α, the TNF-α, IFN-γ, IFN-α and the vitamin may be treated in a ratio of 0.1 to 3:0.1 to 3:0.1 to 3:1,000 to 10,000 (w/v), preferably 0.1 to 3:0.1 to 3:0.1 to 3:1,000 to 6,000 (w/v), more preferably 1:1:0.1 to 3:4,000 to 6,000 (w/v), much more preferably 1:1:0.1 to 3:4,000 to 5,500 (w/v), and even more preferably 1:1:2:5,000 (w/v).

The primed stem cells of the present invention may exhibit at least one effect of alleviating atopic dermatitis selected from the group consisting of reduction of mast cells, reduction of total IgE, increased production of IgG2a, and reduction of histamine. In addition, when the primed stem cells of the present invention are treated in combination with the regulatory T cells, it is possible to achieve the effect of reducing total IgG1 and increasing IgG2a, and as a result, shifting to Th1 is achieved and the immune imbalance is resolved, thereby achieving an atopic dermatitis therapeutic effect.

### MODES FOR THE INVENTION

Hereinafter, the present invention will be described in more detail through Examples. These Examples are just illustrative of the present invention, and it will be apparent to those skilled in the art that it is not interpreted that the scope of the present invention is limited to these Examples.

### Example 1. Culture of Stem Cells and Selection of Candidate Substances

In a 37° C. and 5% CO₂ incubator, mesenchymal stem cells in a storage state (LN₂ tank storage) were thawed and cultured, and at this time, the cells were cultured in a medium (DMEM, alpha-MEM) containing 10% FBS or 4% hPL until proliferated to a cell confluence of about 80%. The cultured mesenchymal stem cells were seeded in a 100 mm dish and treated with candidate substances for enhancing the functions of the mesenchymal stem cells for 24 hours, and then the concentrations of primary candidate substances for enhancing the functions were set.

TNF-α, IFN-γ, and IFN-α were selected as the primary candidate substances, and in order to confirm the lowest effective concentration capable of increasing the expression of TNFα-stimulated gene-6 (TSG6) and indoleamine 2,3-dioxygenase (IDO) to confirm the enhanced functions, the stem cells were treated with 5, 10, and 20 ng/ml of TNF-α, 5, 10, and 20 ng/ml of IFN-γ, and 10, 20, and 40 ng/ml of IFN-α, respectively, and changes in expression of TSG6 and IDO were confirmed.

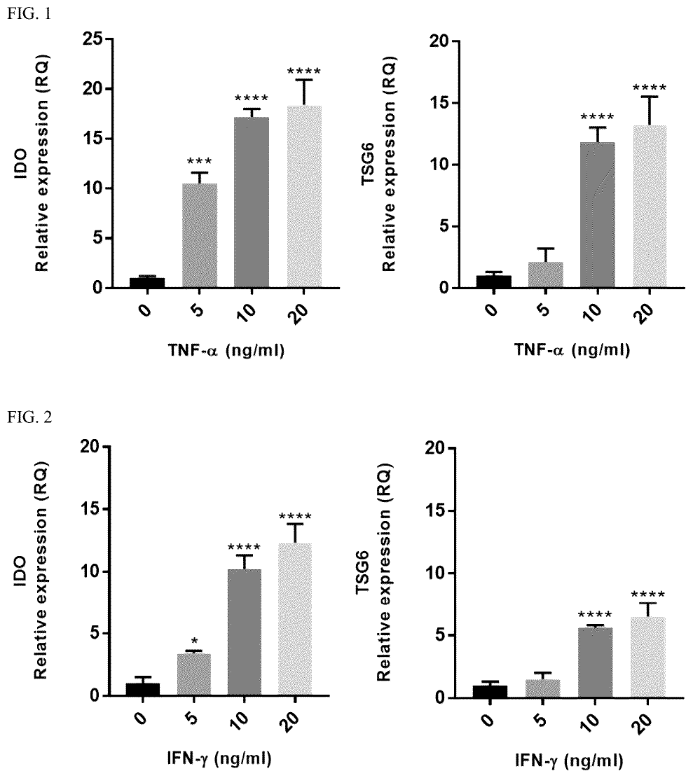

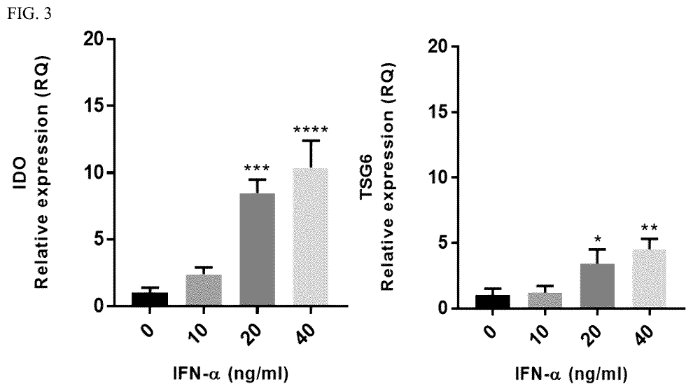

The IDO was known as an immunomodulatory factor that inhibited the proliferation of immune cells such as T cells by converting tryptophan, which was essential for T cell proliferation, into kynurenine, and the TSG6 was known as an anti-inflammatory regulator secreted from mesenchymal stem cells. After culturing the stem cells, total RNA was isolated using TRIzol (Invitrogen), and cDNA was synthesized from total RNA using PrimeScript™RT reagent Kit with gDNA Eraser (TaKaRa), and qRT-PCR was performed. The results of TSG6 and IDO according to the concentration of each candidate substance were illustrated in FIGS. 1, 2 and 3.

As illustrated in FIGS. 1 to 3, 10 ng/ml of TNF-α, 10 ng/ml of IFN-γ, and 20 ng/ml of IFN-α were confirmed as the lowest effective concentrations capable of inducing significantly increased expression of both TSG6 and IDO, and thereafter, the experiment was performed based on the corresponding concentrations.

### Example 2. Selection of Combination for Enhancing Functions of Stem Cells

Through Example 1, in order to search for more function-enhanced compositions based on the candidate substances and lowest effective concentrations identified to induce the enhanced functions of stem cells, selected candidate substances and combined concentrations thereof were set as shown in Table 1. Mesenchymal stem cells were treated with the candidate combinations described in Table 1 below for 24 hours, and the stem cells were cultured as in Example 1, and then total RNA was isolated using TRIzol (Invitrogen). Thereafter, qRT-PCR was performed by synthesizing cDNA from total RNA using the PrimeScript™RT reagent Kit with gDNA Eraser (TaKaRa).

TABLE 1
Experimental Group

| | |
|---|---|
| 0 | Control (Untreated group) |
| 1 | TNF-α (10 ng/ml) |
| 2 | IFN-γ (10 ng/ml) |
| 3 | IFN-α (20 ng/ml) |
| 4 | TNF-α (10 ng/ml) + IFN-γ (10 ng/ml) |
| 5 | TNF-α (10 ng/ml) + IFN-α (20 ng/ml) |
| 6 | IFN-γ (10 ng/ml) + IFN-α (20 ng/ml) |
| 7 | TNF-α (10 ng/ml) + IFN-γ (10 ng/ml) + IFN-α (20 ng/ml) |

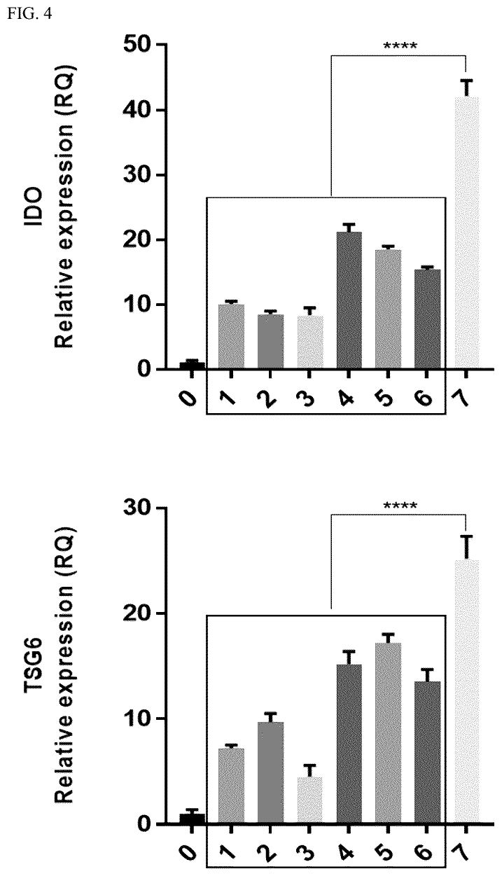

Through this, changes in the expression of IDO and TSG6, which were representative factors of immune and anti-inflammatory regulation, were confirmed, and the results were illustrated in FIG. 4.

As illustrated in FIG. 4, compared to an untreated control group 0, increased expression of IDO and TSG6 was confirmed in groups treated with all single candidate substances, TNF-α, IFN-γ, and IFN-α (Experimental Groups 1 to 3), but in Experimental Groups 4 to 6 in which these candidate substances were combined, a more significant increase in expression of IDO and TSG6 was confirmed compared to single candidate substance treated groups. In particular, Experimental Groups 4 and 5, in which TNF-α was treated with IFN-γ or IFN-α, excellent effects were exhibited compared to Experimental Group 6, in which only IFN-γ and IFN-α were combined, and surprisingly, in a treatment group in which all three candidate substances were combined, a function enhancing effect which was twice or more increased than that of a combination of two substances was confirmed.

Therefore, through the results, when the stem cells were treated with a combination of TNF-α (10 ng/ml)+IFN-γ (10 ng/ml)+IFN-α (20 ng/ml), it was confirmed that very significantly enhanced immunity and anti-inflammatory functions of the stem cells could be achieved.

### Example 3. Confirmation of Function Enhancing Effect of Stem Cells According to Addition of Vitamins

### 3.1 Confirmation of Function Enhancing Effect of Stem Cells According to Addition of Vitamin Alone

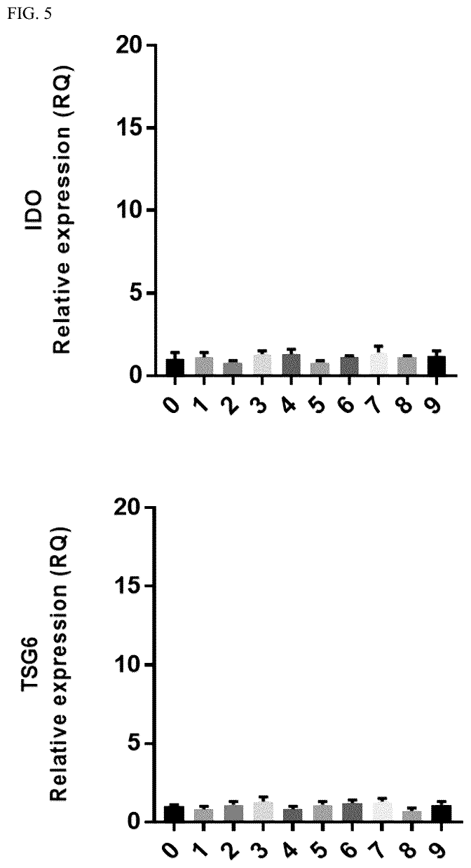

Vitamins were known to improve the proliferative ability of stem cells and maintain the stemness of stem cells when added during a stem cell culture process. Accordingly, various vitamins were treated in the stem cell culture process, and experiments were performed to confirm whether or not vitamins could achieve anti-inflammatory and immune function enhancing effects of stem cells. Specifically, the stem cells were treated with various types of vitamins shown in Table 2 below according to the method described in Example 1, and the results of confirming changes in expression of IDO and TSG6 according to treatment with each candidate substance were illustrated in FIG. 5.

TABLE 2
Experimental Group

| | |
|---|---|
| 0 | Control (Untreated group) |
| 1 | Vitamin A 10 ug/ml |
| 2 | Vitamin B1 50 ug/ml |
| 3 | Vitamin B2 5 ug/ml |
| 4 | Vitamin B3 50 ug/ml |
| 5 | Vitamin B5 50 ug/ml |
| 6 | Vitamin B6 50 ug/ml |
| 7 | Vitamin B12 50 ug/ml |
| 8 | Vitamin D2 10 ug/ml |
| 9 | Vitamin D3 10 ug/ml |

As illustrated in FIG. 5, the vitamin-treated groups did not show significant effects on the changes in expression of IDO and TSG6 involved in the anti-inflammatory and immunomodulatory functions of the stem cells.

### 3.2 Confirmation of Function Enhancing Effect of Stem Cells According to Combination of Vitamin and TNF-α+IFN-γ+IFN-α

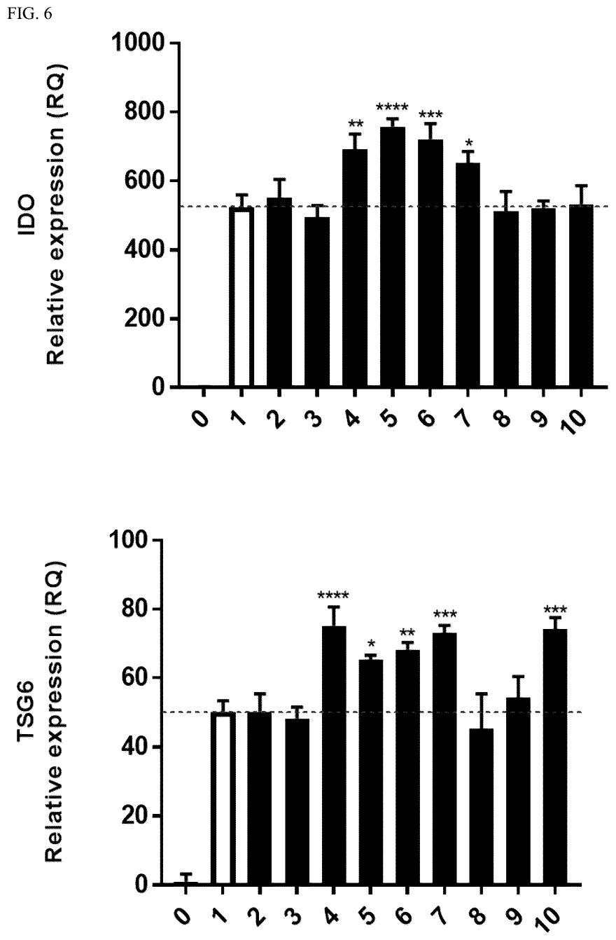

As described above, when vitamins, which did not show an effect of enhancing the anti-inflammatory and immunomodulatory functions of the stem cells as a single substance, were added to a combination of function-enhanced substances identified in Example 2, in order to confirm whether or not to exhibit a synergistic effect, as shown in Table 3 below, each vitamin was added to TNF-α (10 ng/ml)+IFN-γ (10 ng/ml)+IFN-α (20 ng/ml), changes in expression of IDO and TSG6 according to each combined treatment were confirmed, and the results were shown in FIG. 6.

TABLE 3
Experimental Group

| | |
|---|---|
| 0 | Control (Untreated group) |
| 1 | TNF-α (10 ng/ml) + IFN-γ (10 ng/ml) + IFN-α (20 ng/ml) |
| 2 | TNF-α (10 ng/ml) + IFN-γ (10 ng/ml) + IFN-α (20 ng/ml) + |
| | vitamin A 10 ug/ml |
| 3 | TNF-α (10 ng/ml) + IFN-γ (10 ng/ml) + IFN-α (20 ng/ml) + |
| | vitamin B1 50 ug/ml |
| 4 | TNF-α (10 ng/ml) + IFN-γ (10 ng/ml) + IFN-α (20 ng/ml) + |
| | vitamin B2 5 ug/ml |
| 5 | TNF-α (10 ng/ml) + IFN-γ (10 ng/ml) + IFN-α (20 ng/ml) + |
| | vitamin B3 50 mg/ml |
| 6 | TNF-α (10 ng/ml) + IFN-γ (10 ng/ml) + IFN-α (20 ng/ml) + |
| | vitamin B5 50 ug/ml |
| 7 | TNF-α (10 ng/ml) + IFN-γ (10 ng/ml) + IFN-α (20 ng/ml) + |
| | vitamin B6 50 mg/ml |
| 8 | TNF-α (10 ng/ml) + IFN-γ (10 ng/ml) + IFN-α (20 ng/ml) + |
| | vitamin B12 50 ug/ml |
| 9 | TNF-α (10 ng/ml) + IFN-γ (10 ng/ml) + IFN-α (20 ng/ml) + |
| | vitamin D2 10 ug/ml |
| 10 | TNF-α (10 ng/ml) + IFN-γ (10 ng/ml) + IFN-α (20 ng/ml) + |
| | vitamin D3 10 ug/ml |

As shown in FIG. 6, the expression of IDO and TSG6 was not significantly increased in Experimental Groups 2, 3, 8, and 9 compared to Experimental Group 1 without vitamin treatment, and in Experimental Group 10, the expression of TSG6 was increased, but the increase in expression of IDO was not confirmed compared to the control group. On the other hand, in the case of vitamin B2 (Experimental Group 4), vitamin B3 (Experimental Group 5), vitamin B5 (Experimental Group 6), and vitamin B6 (Experimental Group 7), it was confirmed that the expression of both IDO and TSG6 was increased significantly. It was confirmed that in the case of TSG6, as compared to Experimental Group 1 treated with TNF-α+IFN-γ+IFN-α, in Experimental Groups 4, 5, 6, and 7, the expression was increased by 49%, 30%, 35%, and 45%, respectively, and in the case of IDO, as compared to Experimental Group 1, in Experimental Groups 4, 5, 6, and 7, the expression was increased by 25%, 37%, 31%, and 18%, respectively.

As a result, in the case of treating stem cells with vitamins alone, the anti-inflammatory and immunomodulatory ability of stem cells were not improved, but in the case of treating a combination of TNF-α+IFN-γ+IFN-α and vitamin B2, vitamin B3, vitamin B5 or vitamin B6, the effect of TNF-α+IFN-γ+IFN-α may be further enhanced.

### Example 4. Confirmation of Changes in Expression of ICAM1 and VCAM

ICAM1 and VCAM may exhibit effects on various immune and inflammatory-related diseases by inhibiting the proliferation of T cells and inducing death of T cells through T cell binding to reduce excessive immune and inflammatory responses. Accordingly, mesenchymal stem cells were treated for 24 hours with the same Experimental Group described in Table 3 of Example 3 above, and the stem cells were cultured as in Example 1, and then total RNA was isolated using TRIzol (Invitrogen). Thereafter, qRT-PCR was performed by synthesizing cDNA from total RNA using the PrimeScript™RT reagent Kit with gDNA Eraser (TaKaRa).

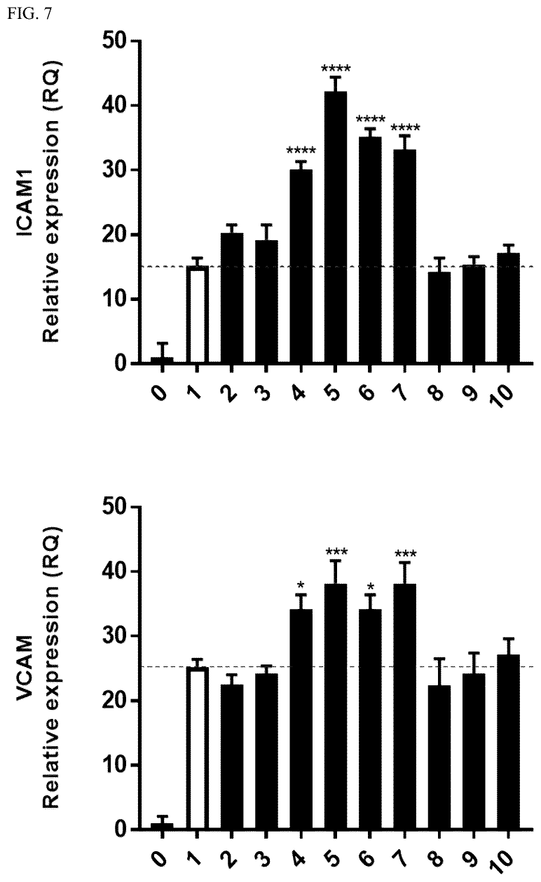

In order to confirm the enhancement of the effect of stem cells for treating immune diseases and inflammatory diseases, the expression of ICAM1 (Intercellular Adhesion Molecule 1) and VCAM (vascular cell adhesion molecule), which were adhesion factors expressed on the surface of stem cells, were confirmed, and the results were illustrated in FIG. 7.

As illustrated in FIG. 7, it was confirmed that in the case of ICAM1, compared to Experimental Group 1 treated with TNF-α+IFN-γ+IFN-α, in Experimental Groups 4, 5, 6, and 7, the expression was increased by 45%, 100%, 35%, and 45%, respectively, and in the case of VACM, compared to Experimental Group 1, in Experimental Groups 4, 5, 6, and 7, the expression was increased by 52%, 69%, 52%, and 69%, respectively.

Therefore, when the stem cells were treated with a combination of TNF-α+IFN-γIFN-α and vitamin B2, vitamin B3, vitamin B5 or vitamin B6, the expression of ICAM1 and VCAM was significantly increased, and as a result, the immunomodulatory activity and the anti-inflammatory effect of the stem cells may be promoted.

### Example 5. Confirmation of Inflammation and Immune Regulation Ability

It was confirmed whether the combination of priming conditions identified in Example 4, which enhanced immune and inflammatory regulators such as IDO, TSG6, ICAM-1, and VCAM, actually inhibited inflammatory and immune responses under conditions for inducing excessive immune responses, and exhibited a superior effect to cMSCs whose functions were not enhanced. Hereinafter, cMSCs were treated with the combination of TNF-α+IFN-γIFN-α and vitamin B6, Experimental Group 7 in Table 3, and MSC primed with the combination was named ‘Primed clonal Mesenchymal Stem Cell 2 (pcMSC2)’.

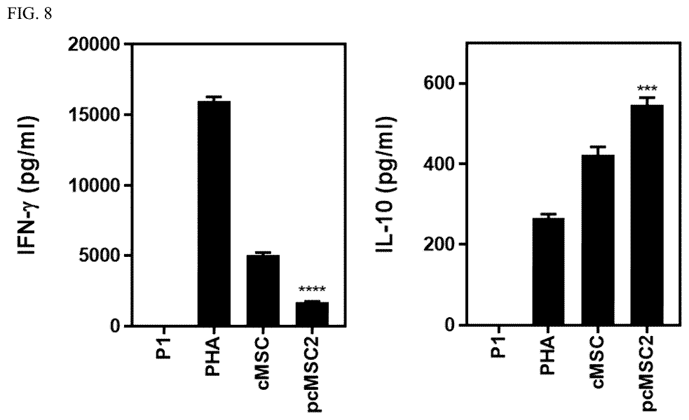

A phytochemagglutinin (PHA)-stimulated lymphocyte culture experiment was a method for inducing excessive immune activity by a method of using a reagent PHA for inducing cell division promotion of lymphocytes. Peripheral blood mononuclear cells (PBMCs, peripheral blood cells) were dispensed at 2×10⁵ cells per well in a 96-well plate, stimulated with 1 μg/ml of PHA, and co-cultured with 5×10⁴ cells of cMSC or pcMSC2 for 4 days. After the culture was completed, the supernatant was collected and inflammatory cytokine, IFN-gamma, and anti-inflammatory cytokine, IL-10, were confirmed through ELISA, and the results were illustrated in FIG. 8. As a negative control group, a group not stimulated with PHA was used, and was denoted as P1.

As illustrated in FIG. 8, it was confirmed that when stimulated with PHA, IFN-gamma increased significantly to 16,013 pg/ml, but when co-cultured with cMSC, IFN-gamma decreased to 5,048 pg/ml, and when co-cultured with pcMSC2, IFN-gamma significantly decreased to 1,700 pg/ml. In addition, when comparing cMSC and pcMSC2, it was confirmed that the function-enhanced pcMSC2 showed a more significant decrease in IFN-gamma by about 66% than cMSC. In the case of the anti-inflammatory cytokine IL-10, an increase in IL-10 was confirmed in both cMSC and pcMSC2, and in particular, under co-culture conditions with pcMSC2, which was function-enhanced compared to cMSC, about 22% more IL-10 was secreted.

### Example 6. Isolation and Culture of Regulatory T Cells (Tregs)

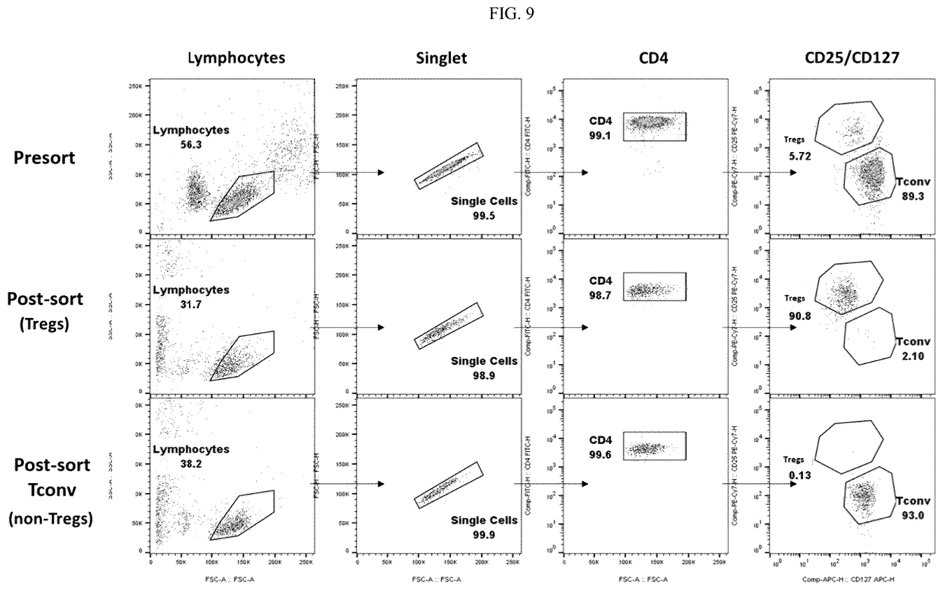

Cord blood mononuclear cells purchased from StemExpress (CA, USA) were thawed, and then only CD4+ cells were enriched and isolated using microbeads (Miltenyi) to recognize human CD4. The CD4+ cell isolation process was performed according to the manufacturer's protocol. Briefly, total cord blood mononuclear cells were mixed with CD4 microbeads, and then only CD4+ cells were purified using a magnetic field column. In order to sort-purify regulatory T cells from the isolated CD4⁺ cells, staining was performed for 20 minutes at 4° C. under a light-protected condition using anti-CD4 FITC (Tonbo), anti-CD25 BV785 (Biolegend), and anti-CD127 APC (Biolegend) antibodies and a flow cytometry cell sorter (BD FACS Melody). The sorting strategy to isolate the regulatory T cells (CD4⁺CD25^{+/high}CD127^{low}, 4-6%) using flow cytometry and purity of the isolated regulatory T cells on day 0 were illustrated in FIG. 9.

Cord blood-derived regulatory T cells (3×10⁵) obtained by the flow cytometry cell sorter were suspended in a 200 μl medium and cultured in a 96-well flat bottom plate. To activate regulatory T cells through CD3 and CD28, MACS GMP T cell TransAct (Miltenyi) was added, and the culture medium was used with an AIM-V media (Gibco) medium containing 5% Human Serum AB (GEMCELL), 500 units/mL penicillin (Gibco), 500 mg/mL streptomycin (Gibco), 1.46 mg/mL Glutamine (Gibco), 300 units/mL recombinant human IL-2 (R&D system), 2 μM BHKps25 (Phosphorothioate-backboned oligonucleotide, TriLink), and 2 ng/ml human TGF-β1 (Cell Genix Inc.). 100 nM rapamycin (Sigma-Aldrich) was added for 3 to 5 days (72 hours) after the start of culture.

To re-stimulate the regulatory T cells on day 6 or 7 of culture, CD4⁻ cells derived from the same donor treated with mitomycin C (Sigma-Aldrich) were added at 1:10 ratio with 50 ng/ml of anti-CD3 antibody (Clone OKT3, Miltenyi). The expanded number of regulatory T cells derived from the cord blood on 17 to 19 days was increased, ranging from 270 to 400 times compared to day 0, depending on the donor.

### Example 7. Identification of Properties of Cord Blood-Derived Regulatory T Cells

To determine whether the characteristics of isolated cord blood-derived regulatory T cells were retained for about 2 weeks of culture and to determine whether the cells were differentiated into other types of cells, a phenotype analysis was performed to identify markers of regulatory T cells and differentiated T cells through flow cytometry on day 12 of culture.

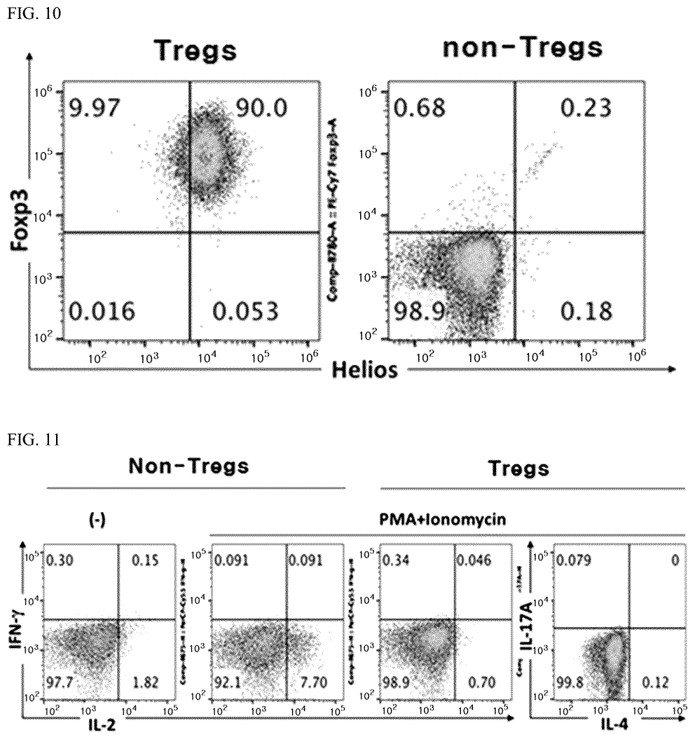

It is known that regulatory T cells maintain immune tolerance function through the expression of specific transcription factors Foxp3 and Helios. To confirm it, some cells were obtained during culture and stained using the corresponding antibodies. Changes in the expression of the markers were observed, and the results were illustrated in FIG. 10.

As illustrated in FIG. 10, it was confirmed that 90% of the cord blood-derived regulatory T cells expressed all the corresponding transcription factors on day 12. On the other hand, most of the non-Treg cells used as a control did not express Foxp3 and Helios.

Additionally, the level of secreted cytokines was measured to confirm the cultured regulatory T cells did not differentiate into other types of cells during the culture period. The regulatory T cells were stimulated to secrete cytokines by treating them with PMA/Ionomycin (Biolegend) for 4 hours, along with a protein transfer inhibitor (Brefeldin A) to prevent the leakage of the cytokines into the culture medium. Since the regulatory T cells were known not to secrete any cytokines, the level of each cytokine was measured to identify differentiation into each lineage of T helper cells, IL-2 for T helper 1 cell (Th1) and natural killer cell (NK cell); IL-4 for IFN-γT helper 2 cell (Th2); IL-17A for T helper 17 cells (Th17). The results were illustrated in FIG. 11.

As illustrated in FIG. 11, the expanded regulatory T cells derived from cord blood had less than 1% of the expression of IL-2, IL-4, IL-17A, and IFN-γ on day 12. This indicates that these cells did not exhibit plasticity of differentiation into Th1, Th2, NK cells, and Th17. Therefore, it has been confirmed that the cord blood-derived regulatory T cells, which were sort-purified through a flow cytometry cell sorter, have maintained their unique functions as regulatory T cells without transforming into other types of cells during the 12-day culture period. These cells were utilized in the experiment.

### Example 8. Combined Administration Effect Using Atopic Dermatitis Animal Model

The effect of treating atopic dermatitis was confirmed by co-administering pcMSC2 in which the function enhancing effect was confirmed in Example 5, and regulatory T cells isolated in Example 6.

### 8.1 Construction of Atopic Dermatitis Model and Administration Protocol

For an atopic dermatitis test, female 6-week-old specific pathogen-free (SPF) BALB/c mice (15 to 20 g) were supplied and used by SLC, Inc (Shizuoka, Japan), and kept in an acclimatization period for 1 week, and then the experiment was performed. At the Life Science Research Institute of Inha University College of Medicine, the experiment was performed while a breeding environment was maintained to freely consume sterilized distilled water and a solid feed at a temperature of 22 to 25° C., humidity of 40 to 60%, and in lighting of 150 to 300 Lux in 12-hour shifts day and night. 7-week-old female BALB/c mice were administered with a mixture of ovalbumin (OVA) 50 μg/50 μl (PBS) and Alum Adjuvant 4 mg/100 μl intraperitoneally and subcutaneously once a week total 3 times for 3 weeks to perform the immunization. Since the mixture was administered to the same area all three times to cause inflammation unrelated to the corresponding disease, the mixture was administered to different areas, respectively. After 3 weeks of OVA+alum administration, a 1.2×1.2 cm sterile gauze was soaked with 60 μg/60 μl (PBS) of OVA and attached to the shaved skin 2 to 3 times a week for 2 weeks to induce atopic dermatitis. The experimental animal was calibrated on days 13 and 14 after induction, and then injected with PBS, cell stabilizer, pcMSC2, regulatory T cells, and pcMSC2+ regulatory T cells using a 1 ml syringe from BD equipped with a 26½ gauge needle, respectively.

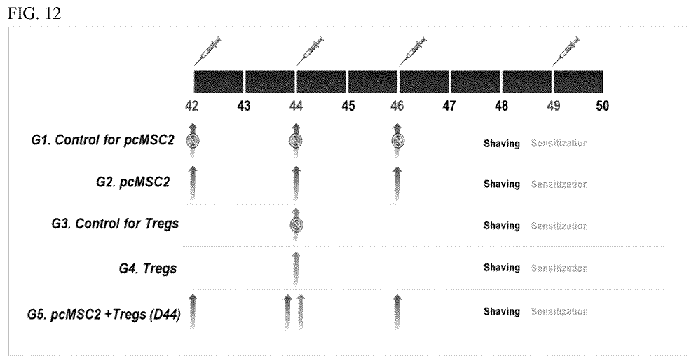

The administration protocol was illustrated in FIG. 12, in which pcMSC administration was indicated by a blue arrow and regulatory T cell administration was indicated by a red arrow. As illustrated in FIG. 12, pcMSC2 was first administered on day 42 and administered intravenously a total of 3 times at 2-day intervals, and administered at 1.66×10⁵/200 μl three times each, for a total of 5.0×10⁵/600 μl. In a Tregs-alone administered group, Tregs were administered a single dose of 1.0×10⁶/250 μl on day 44, and in a pcMSC2+Tregs combined administered group, a total of 1.0×10⁶/250 μl of regulatory T cells was further intradermally administered on day 44 in addition to the pcMSC2 administration protocol (D44). Since the intradermal administration may induce skin trauma if 50 μl or more per area was administered, 50 μl of cells suspended in 250 μl was injected over five areas. The experimental animals in a Veh. group were divided into two groups, Veh. (pcMSC2 control) and Veh. (Tregs control), according to an administration method, and administered intradermally with PBS and administered intravenously with a cell stabilizer, respectively.

After two days, the lesion area was shaved again, and then a 1.2×1.2 cm-sized sterile gauze soaked in OVA 60 μg/60 μl (PBS) was attached to the shaved back skin 3 to 4 times a week for 2 weeks to re-induce atopic dermatitis (boosting step).

To confirm the effect on atopic dermatitis, blood samples were obtained in the following manner. All of the experimental animals were anesthetized using isoflurane on approximately day 10 (day 59 of the experiment) after administration of the cell stabilizer or test substance, and then the abdomen was incised to expose the posterior vena cava and blood was collected. The collected blood was contained in a serum separate tube and centrifuged at 3,000 rpm/15 min at 4° C., and the then contained in a 1.5 ml e-tube and stored in a −70° C. deep freezer.

### 8.2 Confirmation of Changes in Skin Lesion by Combined Administration

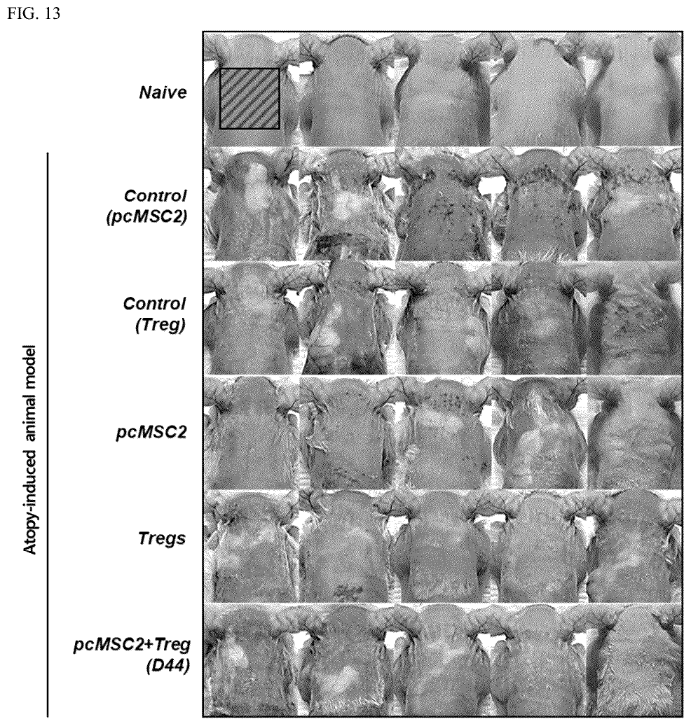

PcMSC2 and regulatory T cells were administered to the experimental animals that induced atopic dermatitis in Example 8.1 using the same protocol as above, and the status of the skin lesion was confirmed on day 59 of the experiment, and the results were illustrated in FIG. 13.

As illustrated in FIG. 13, after test substances were injected into experimental animals that induced atopic dermatitis, the presence or absence of wounds, blood stains, and dead skin cells caused by the disease over the skin was observed. As a result, it was confirmed that the skin was more effectively alleviated in a group co-administered with pcMSC2 and regulatory T cells (D44) than in a single administered group.

### 8.3 Confirmation of Total IgG1 Inhibition Effect and IgG2 Production Effect According to Combined Administration

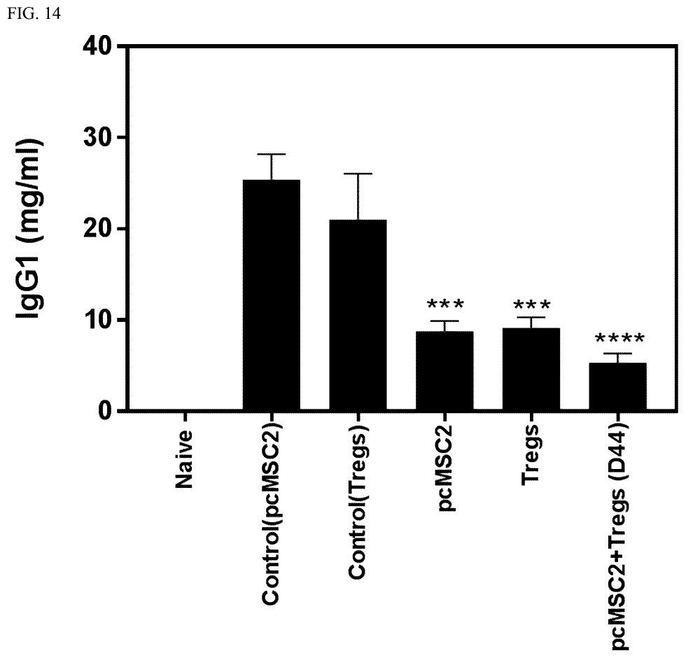

PcMSC2 and regulatory T cells were administered to the atopic dermatitis-induced animal model of Example 8.1 using the same protocol as above, and total IgG1 and IgG2a were compared. To evaluate the effectiveness of atopic dermatitis, the concentrations of total IgG1 and IgG2a in serum samples were measured using an ELISA kit, and the measurement was performed according to the protocol in the kit. Capture antibodies capable of recognizing immunoglobulin were stored on an ELISA plate with a coating buffer at 4° C. overnight, and the next day, after going through a blocking process for 1 hour, the serum was diluted at least 100 times and reacted at room temperature in an amount of 50 μl for 2 hours. Thereafter, the serum was added with a secondary antibody recognizing immunoglobulin, added with a TMB solution as a substrate buffer, reacted for about 15 minutes, and then added with 50 μl of a stop solution. The values were measured at a filter of 450 nm using an ELISA reader. The confirmed results were illustrated in FIGS. 14 to 17.

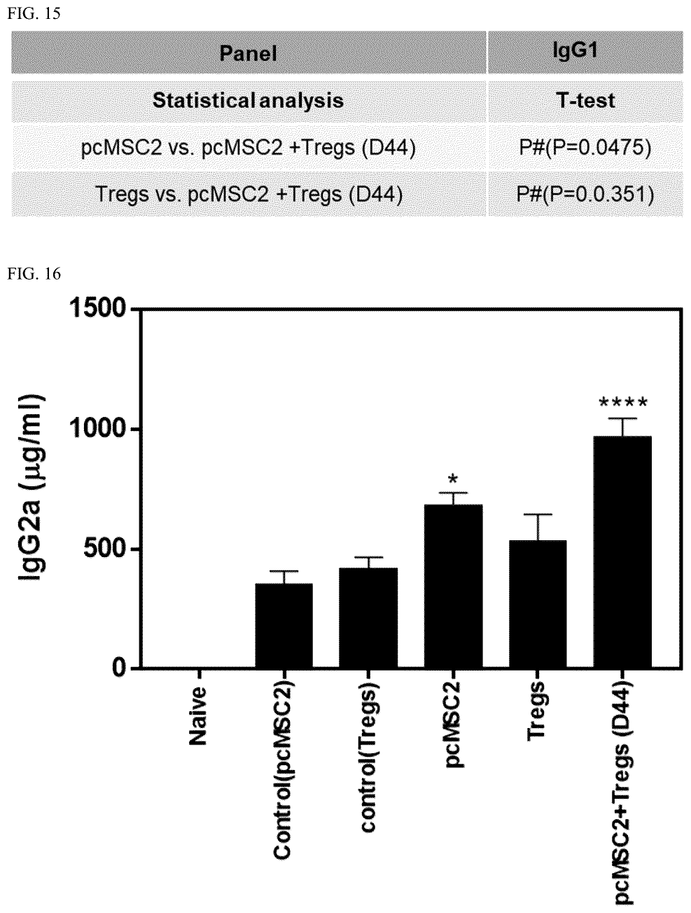

As illustrated in FIGS. 14 and 15, it was confirmed that Total IgG1 was significantly reduced in all test substance-administered groups except Veh., and particularly, reduced with the greatest width when the regulatory T cells were co-injected into pcMSC2.

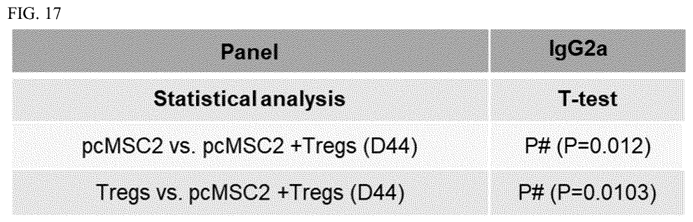

As illustrated in FIGS. 16 and 17, in the case of IgG2a, a pattern opposite to the result of IgG1 was confirmed, but it was confirmed that the levels thereof increased to a significant level in all test substance administered groups except for the regulatory T cell-alone administered group compared with Veh. In addition, compared to the single injection groups of pcMSC2 and regulatory T cells, the levels thereof increased to the greatest width when pcMSC2 was injected in combination with the regulatory T cells. This result indicated that by injecting pcMSC2 and regulatory T cells into a model of atopic dermatitis, a Th2 disease, shifting to Th1 is achieved, thereby controlling immune imbalance, and alleviating atopic symptoms.

As described above, specific parts of the contents of the present invention have been described in detail, and it will be apparent to those skilled in the art that these specific techniques are merely preferred embodiments, and the scope of the present invention is not limited thereto. Therefore, the substantial scope of the present invention will be defined by the appended claims and their equivalents.

1-21. (canceled)

22. A method for preventing or treating atopic dermatitis comprising:
1) administering stem cells treated with TNF-α, IFN-γ, and IFN-α to a subject in need thereof; and
2) administering regulatory T cells to the subject.

23. The method for preventing or treating atopic dermatitis of claim 22, wherein the TNF-α, IFN-γ, and IFN-α are treated in a ratio of 0.1 to 3:0.1 to 3:0.1 to 3 (w/v).

24. The method for preventing or treating atopic dermatitis of claim 22, wherein the stem cells are stem cells further treated with at least one vitamin selected from the group consisting of vitamin B2, vitamin B3, vitamin B5 and vitamin B6.

25. The method for preventing or treating atopic dermatitis of claim 24, wherein the TNF-α, IFN-γ, and IFN-α and the vitamin are treated in a ratio of 0.1 to 3:0.1 to 3:0.1 to 3:100 to 10,000 (w/v).

26. The method for preventing or treating atopic dermatitis of claim 22, wherein the stem cells are administered in combination with the regulatory T cells.

27. The method for preventing or treating atopic dermatitis of claim 22, wherein the stem cells are mesenchymal stem cells derived from fat, bone marrow, placenta, or cord blood.

28. The method for preventing or treating atopic dermatitis of claim 22, wherein the regulatory T cells are regulatory T cells derived from blood, fat, bone marrow, placenta, or cord blood.

29. The method for preventing or treating atopic dermatitis of claim 22, wherein the stem cells are enhanced with at least one selected from the group consisting of expression of TNFα-stimulated gene-6 (TSG6), expression of Indoleamine 2,3-dioxygenase (IDO), expression of Intercellular adhesion molecule 1 (ICAM1), and expression of Vascular cell adhesion molecule (VCAM).

30. A combined administration kit for preventing or treating atopic dermatitis comprising:
compartment 1 containing stem cells treated with TNF-α, IFN-γ, and IFN-α; and
compartment 2 containing regulatory T cells.

31. The combined administration kit for preventing or treating atopic dermatitis of claim 30, wherein the compartment 1 further comprises at least one vitamin selected from the group consisting of vitamin B2, vitamin B3, vitamin B5 and vitamin B6.

32. The combined administration kit for preventing or treating atopic dermatitis of claim 30, wherein the TNF-α, IFN-γ, and IFN-α are contained in a ratio of 0.1 to 3:0.1 to 3:0.1 to 3 (w/v).

33. The combined administration kit for preventing or treating atopic dermatitis of claim 31, wherein the TNF-α, IFN-γ, and IFN-α and the vitamin are treated in a ratio of 0.1 to 3:0.1 to 3:0.1 to 3:100 to 10,000 (w/v).

34. The combined administration kit for preventing or treating atopic dermatitis of claim 30, wherein the stem cells are mesenchymal stem cells derived from fat, bone marrow, placenta, or cord blood.

35. The combined administration kit for preventing or treating atopic dermatitis of claim 30, wherein the regulatory T cells are regulatory T cells derived from blood, fat, bone marrow, placenta, or cord blood.

36. A cosmetic composition for preventing or alleviating atopic dermatitis, comprising stem cells treated with TNF-α, IFN-γ, and IFN-α; and regulatory T cells.

37. The cosmetic composition for preventing or alleviating atopic dermatitis of claim 36, wherein the stem cells are stem cells further treated with at least one vitamin selected from the group consisting of vitamin B2, vitamin B3, vitamin B5 and vitamin B6.

38. The cosmetic composition for preventing or alleviating atopic dermatitis of claim 36, wherein the TNF-α, IFN-γ, and IFN-α are treated in a ratio of 0.1 to 3:0.1 to 3:0.1 to 3 (w/v).

39. The cosmetic composition for preventing or alleviating atopic dermatitis of claim 37, wherein the TNF-α, IFN-γ, and IFN-α and the vitamin are treated in a ratio of 0.1 to 3:0.1 to 3:0.1 to 3:100 to 10,000 (w/v).

40. The cosmetic composition for preventing or alleviating atopic dermatitis of claim 36, wherein the stem cells are mesenchymal stem cells derived from fat, bone marrow, placenta, or cord blood.

41. The cosmetic composition for preventing or alleviating atopic dermatitis of claim 36, wherein the regulatory T cells are regulatory T cells derived from blood, fat, bone marrow, placenta, or cord blood.
